(19) United States
(12) Patent Application Publication
(10) Pub. No.: US 2026/0168982 A1
(43) Pub. Date: Jun. 18, 2026

(54) HYPERSPECTRAL NEMATODE DETECTION OF ORGANOPHOSPHATE SITE CONTAMINATION

(71) Applicant: RTX BBN Technologies, Inc., Cambridge, MA (US)

(72) Inventors: Sarah Nicole Jeter, Henrietta, NY (US); Miles Thomas Rogers, Mansfield, MA (US); Kemper Talley, Knoxville, TN (US)

(21) Appl. No.: 19/419,925

(22) Filed: Dec. 15, 2025

Related U.S. Application Data
(60) Provisional application No. 63/734,512, filed on Dec. 16, 2024.

(51) Int. Cl. G01N 33/24; G06T 7/00

(52) U.S. Cl. CPC G01N 33/24; G06T 7/0012; G06T 2207/20081; G06T 2207/20084

(57) ABSTRACT

In stark contrast to traditional methods, embodiments of the disclosure observe and determine soil contamination levels by leveraging the natural sensitivity of soil microbial life (nematodes, algae, yeasts, bacteria, fungi, etc.) to contaminants within the ecosystem. Embodiments disclosed, use hyperspectral imaging and/or other additional imaging combined with one or more machine learning algorithms to determine biological changes that indicate a contaminant presence, absence, or concentration in the soil.

### CROSS-REFERENCE TO RELATED APPLICATIONS

This application claims the benefit of priority to U.S. Application No. 63/734,512, filed Dec. 16, 2024, the disclosure of which is incorporated herein by reference in its entirety.

### BACKGROUND

The present disclosure relates to innovative methods and systems for detecting soil contamination, with an emphasis on organophosphate compounds. Organophosphate compounds, prevalent in pesticides and chemical warfare agents, pose significant health risks and require effective monitoring to ensure environmental and human safety.

Soil contamination detection involves identifying harmful substances within soil samples, often using chemical, biological, or physical methods. The field encompasses various techniques to monitor and assess the presence of contaminants such as pesticides, heavy metals, and chemical agents. Applications include environmental monitoring, agricultural safety, and emergency response, where accurate detection ensures the protection of human health and the environment.

In environmental monitoring, the goal is to identify and quantify contaminants to prevent ecological damage and ensure compliance with safety regulations. Agricultural safety focuses on detecting harmful substances in soil to protect crops and food supplies. Emergency response applications require rapid detection of chemical agents to safeguard first responders and the public in hazardous situations. These goals necessitate reliable, efficient, and sensitive detection methods that can operate in diverse and challenging environments.

Current methods for detecting contamination in soil often require extensive manual processes. These methods demand skilled personnel to achieve accurate results, which can be time-consuming and resource intensive. The need for rapid and reliable detection in field conditions remains unmet, particularly for ensuring the safety of first responders, site inspectors, and field workers.

Organophosphates, due to their widespread use, potential for harm, and multiplicity of compounds often require precise detection techniques. Traditional methods, such as gas chromatography and mass spectrometry, while accurate, are not always feasible for on-site analysis due to their complexity and the need for controlled laboratory settings. Traditional methods are further hindered by long supply chains that need to provide consumables such as wetting agents, dyes, gases, and the like, further decreasing portability and limiting potential field use.

Existing sensors designed for in-situ detection face challenges related to reliability and sensitivity, especially when targeting organophosphates. These limitations hinder their effectiveness in providing timely and accurate assessments of contamination. Other detecting techniques, such as those that rely on bacteria, require the appropriate support equipment for detection and/or require the creation and introduction of their own strain of bacteria. This further increases the cost and complexity of deploying such techniques in the field.

Thus, there is a clear need for advanced detection technologies that offer rapid, reliable, and sensitive analysis of organophosphate contamination in the field, ensuring the safety and protection of both the environment and first responders.

### SUMMARY

Embodiments disclosed reflect the development of a more efficient and autonomous detection process capable for use by minimally trained personnel effectively addressing the aforementioned shortcomings and enhancing the capability to qualitatively and quantitatively detect harmful substances, including organophosphates, in various environments.

An example embodiment may be found in a system for determining soil contaminant levels. The system comprises: at least one processor; and, at least one memory device that stores an application. The application adapts the at least one processor to: acquire one or more hyperspectral images from a soil sample provided to a hyperspectral imaging system; isolate one or more images of nematodes, present in the soil sample; pass the images of the one or more isolated nematodes into an input layer of a machine learning model trained from a set of known nematode parameters responsive to at least one soil contaminant; identify and measure bioindicator parameters of the one or more isolated nematodes; determine a concentration of the at least one soil contaminant; and, transmit an indicator corresponding to the concentration of the at least one soil contaminant. In certain embodiments, the soil contaminant is at least one of: an organophosphate, an herbicide, a pesticide, a fungicide, and a heavy metal. In other embodiments, the hyperspectral imaging system includes at least one lighting source configured to induce autofluorescence in nematodes. In still other embodiments, the hyperspectral image is generated in the visible and near-infrared wavelengths. In certain additional embodiments, the hyperspectral image is generated using wavelengths above 500 nm. In further embodiments, the nematode parameters include at least one of: presence or absence of hook shaped morphology; nematode count; nematode fluorescence strength and/or color; and nematode motility. In still other embodiments, the machine learning model is a convoluted neural network trained on a training set of nematode parameters. In additional embodiments, the soil sample is imaged in situ. In still other embodiments the system is further configured to measure at least one or more additional bioindicators. In some embodiments, the at least one or more additional bioindicators are selected from the group of: amoebas, fungi, algae, yeasts, and bacteria.

Some embodiments of the disclosure are in the form of a method. In such embodiments the disclosure may present as a method of detecting soil contamination levels comprising: a) providing a soil sample to a hyperspectral imaging system; b) generating at least one hyperspectral image with the hyperspectral imaging system; c) isolating one or more nematode images in the soil sample; d) passing the images of the one or more isolated nematodes into an input layer of a machine learning model trained from a set of known nematode parameters responsive to a soil contaminant; e) identifying and measuring the nematode parameters; f) determining a concentration of the at least one soil contaminant; and, g) transmitting an indicator corresponding to the concentration of the soil contaminant. In certain embodiments, the soil sample is provided to the hyperspectral imaging system via a sample scoop deployed from an autonomous decontamination system. In still other embodiments, the machine learning model is a convolutional neural network.

Some embodiments may take the form of a non-transitory computer readable medium comprising instructions that adapt at least one processor to: acquire one or more hyperspectral images from a soil sample provided to a hyperspectral imaging system; isolate one or more images of nematodes present in the soil sample; pass the one or more images of isolated nematodes into an input layer of a machine learning model trained from a set of known nematode parameters responsive to a soil contaminant; identify and measure nematode parameters in the images of the isolated nematodes; determine a concentration of the at least one soil contaminant; and, transmit an indicator corresponding to the concentration of the soil contaminant. In additional embodiments the machine learning model is an artificial neural network.

Additional features and advantages are realized through the techniques of the present disclosure. Other embodiments and aspects of the disclosure are described in detail herein and are considered a part of the claimed disclosure. For a better understanding of the disclosure with the advantages and the features, refer to the description and to the drawings.

### BRIEF DESCRIPTION OF THE SEVERAL VIEWS OF THE DRAWINGS

For a more complete understanding of this disclosure, reference is now made to the following brief description, taken in connection with the accompanying drawings and detailed description, wherein like reference numerals represent like parts:

FIG. 1 presents an image of a representative nematode in the soil.

FIG. 2 presents a global map of nematode abundance per unit area (m²).

FIG. 3 presents an example image of two nematodes (arrowheads) exhibiting “hook” morphology in response to a stressor contrasted with a nematode not exhibiting the morphology.

FIG. 4 presents a comparison of a traditional chemical contamination method (left column) contrasted with an embodiment of the disclosure (right column).

FIG. 5 presents the autofluorescence in 13-day old nematodes measured using a fluorescence spectrophotometer (ex. 340 nm/em 360-600 nm).

FIG. 6 presents a fluorescence scan of the nematode Ascaris lumbricoides at 561 nm excitation.

FIG. 7 provides an example of detection of the nematode Anisakis using measurements ranging from 350-1000 nm.

### DETAILED DESCRIPTION

Embodiments disclosed reflect the development of a more efficient and autonomous detection process for soil contaminants, particularly for organophosphates, capable for use by minimally trained personnel. In general, embodiments of the disclosure can include an imaging system, computer hardware, a power source, and the requisite hardware and software to interconnect the components. In certain embodiments, a hyperspectral imaging system is used to capture one or more still or video images of soil samples. The images are passed through an artificial intelligence algorithm, such as a CNN, and nematodes, if present, are differentially recognized and categorized from background materials (e.g., soil particles, organic matter, other animals, etc.). Nematode morphology and behavior is then measured and categorized in a fashion indicative of the presence of one or more contaminants.

Prior to describing the disclosure in deeper detail, the following terms may be helpful.

As used herein the terms “computer,” “computer system,” “computer module,” and the like refer to a system that may include all the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. Systems may include at least one processor and system memory/data storage structures, which may include random access memory (RAM) and read-only memory (ROM). The at least one processor may include one or more conventional microprocessors and one or more supplementary co-processors such as math co-processors, field-programmable-gate arrays, neural engines, or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, a software application that adapts a controller to perform the methods disclosed herein may be read into a main memory of the at least one processor from a computer-readable medium. The term “computer-readable medium”, as used herein, refers to any medium that provides or participates in providing instructions to the at least one processor of a computer system (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes at least one processor to perform the methods/processes described herein, hard-wired circuitry may be used in place of, or in combination with, software instructions for implementation of the methods/processes of the present invention. Therefore, embodiments of the present disclosure are not limited to any specific combination of hardware and/or software.

Certain aspects of the present disclosure may provide for an application, the application may provide for an interface, e.g., a web-based user interface or other type of network interface to provide known training images for previously unknown conditions. That is, multiple users may assist in training neural network embodiments by providing updated images of confirmed conditions.

As used herein a convolutional neural network (“CNN”) is a network architecture for deep learning that learns directly from data. CNNs are particularly useful for finding patterns in images to recognize objects, classes, and categories. They can also be effective for classifying audio, time-series, and signal data. CNNs use a series of layers, each of which detects different features of an input image. Depending on the complexity of its intended purpose, a CNN can contain dozens, hundreds or even thousands of layers, each building on the outputs of previous layers to recognize detailed patterns.

The general overarching architecture of a CNN is typically divided into three layers: a convolutional layer, a pooling layer, and a fully connected layer. As data passes through these layers, the complexity of the CNN increases, which lets the CNN successively identify larger portions of an image and more abstract features. The convolutional layer is the fundamental building block of a CNN and is where the majority of computations occur. This layer uses a filter or kernel—a small matrix of weights—to move across the receptive field of an input image to detect the presence of specific features. The pooling layer of a CNN is a critical component that follows the convolutional layer. Similar to the convolutional layer, the pooling layer's operations involve a sweeping process across the input image, but its function is otherwise different. The pooling layer aims to reduce the dimensionality of the input data while retaining critical information, thus improving the network's overall efficiency. This is typically achieved through downsampling: decreasing the number of data points in the input. The fully connected layer plays a critical role in the final stages of a CNN, where it is responsible for classifying images based on the features extracted in the previous layers. The term fully connected means that each neuron in one layer is connected to each neuron in the subsequent layer.

The fully connected layer integrates the various features extracted in the previous convolutional and pooling layers and maps them to specific classes or outcomes. Each input from the previous layer connects to each activation unit in the fully connected layer, enabling the CNN to simultaneously consider all features when making a final classification decision. Those of skill in the art appreciate that the above discussion of CNNs does not necessarily contain every parameter or feature but merely provides a broad description encompassing what is a more deeply detailed field.

As used herein, “neural networks” or “artificial neural networks” refer to a specific class of machine learning algorithms. Artificial neural networks are modeled on the human brain, in which thousands or millions of processing nodes are interconnected and organized into layers.

In an artificial neural network, cells, or nodes, are connected, with each cell processing inputs and producing an output that is sent to other neurons. Labeled data moves through the nodes, or cells, with each cell performing a different function. In a neural network trained to identify whether a picture contains a cat or not, the different nodes would assess the information and arrive at an output that indicates whether a picture features a cat.

As used herein, “deep learning” refers to neural networks with many layers. The layered network can process extensive amounts of data and determine the “weight” of each link in the network. For example, in an image recognition system, some layers of the neural network might detect individual features of a face, like eyes, nose, or mouth, while another layer would be able to tell whether those features appear in a way that indicates a face. Likewise, in certain embodiments, deep learning networks may differentiate nematodes from soil particles and identify behaviors and features of the nematodes in response to the presence of and/or quantity of a contaminant.

While the machine learning model used in embodiments of the disclosure is an artificial neural network, it will be understood the machine learning model may take other forms. In embodiments, the learning model may implement linear regression, logistic regression, supervised learning, unsupervised learning, or other suitable machine learning approaches.

As used herein, “hyperspectral imaging” refers to a technique that captures and processes information across a wide spectrum of electromagnetic radiation. Unlike traditional imaging, which captures images in three primary colors (red, green, and blue), hyperspectral imaging collects data in numerous narrow spectral bands. This allows for the identification and analysis of materials based on their spectral signatures, making it useful in applications like agriculture, environmental monitoring, and chemical detection.

As used herein a “hyperspectral imager” or “hyperspectral imaging system” refers to all of the necessary hardware and associated equipment required to generate a hyperspectral image/s. This encompasses all necessary optics, power supplies, communications modules, light sources, etc. An example hyperspectral imager is found in an ORCA-spark CMOS camera. Optics may include standard lenses such as primes, or special lenses such as macro lenses, telephoto lenses, or microscopic lenses as the situation warrants to obtain the images of the bioindicator organisms, such as nematodes.

As used herein, the term “training set,” in the context of a CNN model refers to a collection of labeled data used to teach the model how to recognize patterns and features. This dataset includes input data, such as images, along with corresponding labels or annotations that indicate the desired output. During training, the CNN processes the data, adjusting its internal parameters to minimize the difference between its predictions and the actual labels. This process enables the model to learn and generalize from the data, improving its accuracy in recognizing similar patterns in new, unseen data. Additional terms similar to “training set” may include “training images,” or “training video” which refer to images and video used as the whole or part of a training set.

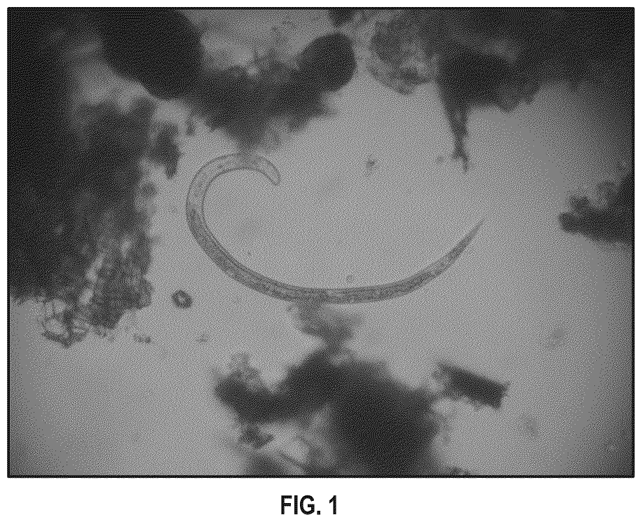

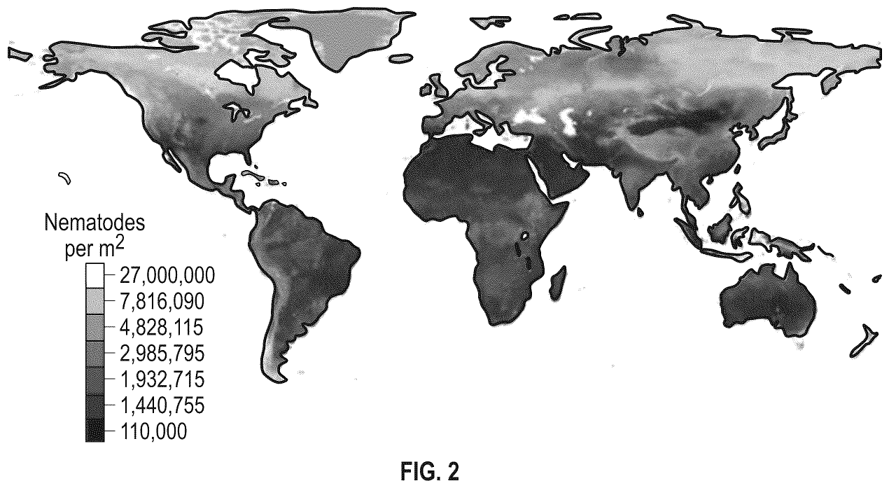

As used herein, the term “nematode” refers to a type of roundworm belonging to the phylum Nematoda. Nematodes play crucial roles in ecosystems, such as decomposing organic matter and controlling pest populations. Some species, however, can cause diseases in plants, animals, and humans. FIG. 1 presents an image of a representative nematode in the soil. These microscopic, unsegmented worms are found in diverse environments, including soil, water, and as parasites in plants and animals. FIG. 2 illustrates a global map of total nematode abundance per unit area (m²).

Nematodes, or roundworms, encompass a vast array of species, each adapted to different environments and roles. Free-living nematodes thrive in soil and aquatic habitats, contributing to nutrient cycling and organic matter decomposition. Parasitic nematodes, on the other hand, can infect plants, animals, and humans, often causing significant agricultural and health issues. For example, root-knot nematodes damage crops by feeding on plant roots, while species like Ascaris lumbricoides infect humans, leading to health complications. Despite their diversity, nematodes share a simple, elongated body structure, allowing them to adapt to various ecological niches and play essential roles in ecosystems worldwide.

Nematodes have a simple, elongated, and cylindrical body structure, typically tapering at both ends. Their bodies are covered by a tough, flexible outer layer called the cuticle, which provides protection and support. Beneath the cuticle lies the epidermis, which secretes the cuticle and maintains its integrity. Internally, nematodes possess a pseudocoelom, a fluid-filled body cavity that acts as a hydrostatic skeleton, aiding in movement. Their digestive system is a straight tube running from the mouth to the anus, with a muscular pharynx that helps ingest food. Nematodes lack a circulatory and respiratory system, relying on diffusion for gas exchange and nutrient distribution. The nervous system is relatively simple, consisting of a nerve ring around the pharynx and longitudinal nerve cords. Nematodes also have sensory structures, such as amphids and phasmids, which help them detect environmental cues. Reproductively, nematodes can be dioecious, with separate male and female individuals, or hermaphroditic. Males often have specialized structures, like spicules, for mating. This basic yet efficient body plan allows nematodes to thrive in diverse environments and fulfill various ecological roles.

The epidermis (skin) of a nematode is highly unusual; it is not composed of cells like other animals, but instead is a mass of cellular material and nuclei without separate membranes. This epidermis secretes a thick outer cuticle which is both tough and flexible. The cuticle is a feature shared with arthropods and other ecdysozoans. As in those other groups, the cuticle is periodically shed during the life of a nematode as it grows, usually four times before reaching the adult stage. The cuticle is the closest thing a roundworm has to a skeleton, and in fact the worm uses its cuticle as a support and leverage point for movement. Long muscles lie just underneath the epidermis. These muscles are all aligned longitudinally along the inside of the body, so the nematode can only bend its body from side to side, not crawl or lift itself. A free-swimming roundworm thus looks rather like it is thrashing about aimlessly.

The muscles are activated by two nerves that run the length of the nematode on both the dorsal (back) and ventral (belly) side. Unlike other animals, where the nerves branch out to the muscle cells, a nematode's muscle cells branch toward the nerves. The ventral nerve has a series of nerve centers along its length, and both nerves connect to a nerve ring and additional nerve centers located near the head.

The head of a nematode has a few tiny sense organs, and a mouth opening into a muscular pharynx (throat) where food is pulled in and crushed. This leads into a long simple gut cavity lacking any muscles, and then to an anus near the tip of the body. Food digested in the gut is not distributed by any specialized vascular system, and neither is there a respiratory system for the uptake or distribution of oxygen. Rather, nutrients and waste are distributed in the body cavity, whose contents are regulated by an excretory canal along each side of the body.

Many nematodes can suspend their life processes completely when conditions become unfavorable; in these resistant states they can survive extreme drying, heat, or cold, and then return to life when favorable conditions return. This is known as cryptobiosis and is a feature nematodes share with rotifers and tardigrades.

When gliding through a relatively densely packed suspension of soil grains the head of a nematode acts as a wedge operated by the forward thrust exerted by the rest of the body. It displaces the grains normally to its own surface and drills a sinusoidal channel through which the rest of the body glides tangentially. Under optimum conditions the speed of the forward progression of the animal is equal to the speed at which the waves of muscular contraction pass backwards relative to the head of the animal; the waves remain stationary relative to the ground and the animal leaves behind it a sinusoidal track whose wavelength and amplitude are the same as those of the muscular waves. The mechanics of the movement are identical with those of a snake gliding through a sinusoidal tube with rigid walls. Conditions such as soil moisture, pH, composition (soil type), and the presence of one or more bio-affective chemicals (e.g., pesticides, herbicides, heavy metals, and the like) can affect mobility through the soil. It is also known that electric and/or magnetic energy can impact orientation and mobility.

Without being bound to a particular theory of operation, it is believed that the abundance and activity of various nematode species in a particular environment will change as the environment changes. In certain embodiments it is by measuring such changes, that it is determined what disruptions the environment may have faced. In certain embodiments nematodes are used to assess the presence of organophosphates in a local environment. The ubiquity and sensitivity of nematodes to environmental change makes them an ideal bioindicator. As illustrated in FIG. 2, nematodes are found in all environments, from tundra to forests to desert, at a rate of 100 s-1000 s per 100 g of dry soil. In addition, the nematode Caenorhabditis elegans (C. elegans) is a common model organism with an abundance of readily available data and protocols.

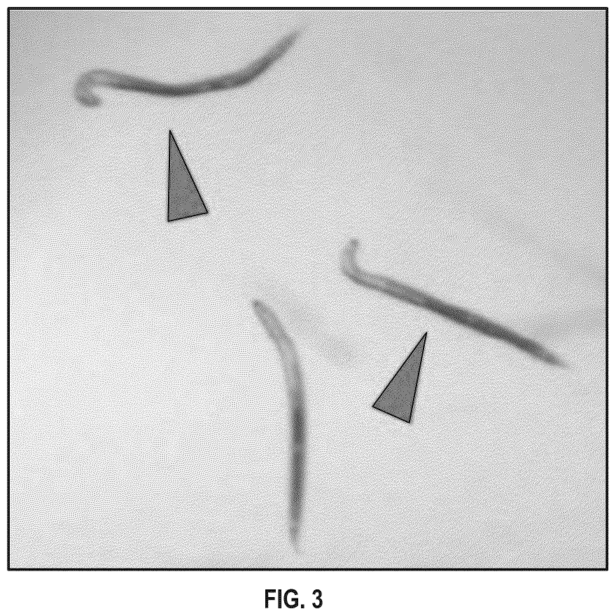

When exposed to stressors, such as herbicides, fungicides, insecticides, etc. nematodes often exhibit a “hook” morphology as seen in FIG. 3 which illustrates two specimens displaying the morphology (arrowheads) contrasted with one specimen that does not.

As used herein, “soil” describes the natural, dynamic medium composed of minerals, organic matter, water, and air. It forms the upper layer of the Earth's crust and serves as a vital resource for plant growth, providing nutrients and a habitat for organisms. Soil develops over time through the weathering of rocks and the decomposition of organic materials, resulting in a complex structure with distinct layers, or horizons. It plays a crucial role in supporting ecosystems, regulating water flow, and influencing climate through carbon storage.

Soil is composed of particles of varying sizes, which influence its texture and properties. The primary particle sizes are: 1. Sand. The largest soil particles, ranging from 0.05 to 2 millimeters in diameter. Sand particles feel gritty and allow for good drainage and aeration but have low nutrient retention. 2. Silt. Medium-sized particles, ranging from 0.002 to 0.05 millimeters. Silt feels smooth and powdery when dry and retains more moisture and nutrients than sand. 3. Clay. The smallest particles, less than 0.002 millimeters in diameter. Clay feels sticky when wet and hard when dry. It has high nutrient retention and water-holding capacity but can lead to poor drainage and aeration. The combination of these particles determines soil texture, affecting the ability of the soil to support plant growth and its overall behavior in the environment. In various embodiments, different soil particle sizes are recognized and differentiated from nematodes.

### Abbreviations

ADS autonomous decontamination system
CNN convolutional neural network
ML machine learning/machine learning model.

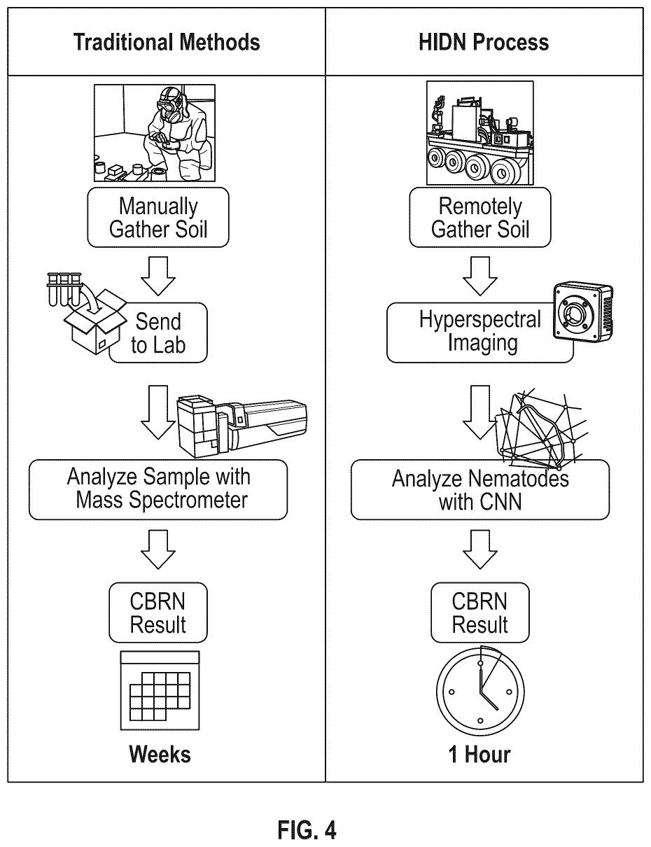

Organophosphates are toxic compounds that result in about 300,000 deaths annually around the globe. Toxicity in humans depends on the type of organophosphate and method of exposure. Organophosphates are primarily used in herbicides and insecticides but have also been weaponized for chemical warfare in certain instances. Traditional means of testing for organophosphates in soil is a tedious, manual process. A sample that is suspected to be contaminated must be collected, dried, crushed, and processed with additional solutions and/or equipment like a centrifuge or mass spectrometer (left column of. FIG. 4). This process induces long delays between sampling and determining whether soil contains organophosphates.

In stark contrast to the traditional methods, embodiments of the disclosure observe and determine soil contamination levels by leveraging the natural sensitivity of soil microbial life to contaminants within the ecosystem. Embodiments disclosed, generally presented in schematic at FIG. 4, right column) use hyperspectral imaging combined with one or more machine learning algorithms to determine biological changes that indicate a contaminant concentration in the soil within an hour or less rather than weeks. In certain embodiments the contaminant concentration may be qualitative (e.g., “more” or “less” present or “absent/present”) rather than strictly quantitative. In still other embodiments a quantitative measurement of concentration may be provided.

Previous studies have used C. elegans as a mammalian neurological model for exposure to acetylcholinesterase inhibitors, finding that the effective concentration, as observed through locomotion changes, to differing levels of toxicity were significantly correlated to LD50 in rats and mice. Importantly, these studies were not based on molecular analyses, but rather on visible observation of nematode behavior—when exposed to organophosphates, the nematodes exhibit significant changes in locomotion, eventually becoming paralyzed. By analyzing these changes, the toxicity effects of various organophosphates can be measured over 4.5 orders of magnitude.

Thus, embodiments of the disclosure can use motility assays to determine the impact of varying organophosphate concentrations such as Dimethylphenylpiperazinium (DMPP) on nematode locomotion. Motility assays will allow for quantification of organophosphate levels in the sampling environment in a dose dependent manner. Imaging of the nematodes can be performed using embodiments and techniques for hyperspectral imaging described herein. In embodiments, images taken over 2 second timepoints for 3 minutes allow for the tracking and measurement of nematode locomotion speed. To generate training sets for embodiments, measurements are performed on nematodes exposed to varying concentration of the target organophosphates. Initial measurements are taken without soil to provide initial clean data for training. Once locomotion impacts are determined, training for the same effect on nematodes in soil is completed.

In certain envisioned embodiments, an ADS vehicle navigates to a location of suspected contamination and uses a robotic arm with a sample scoop/extractor to place soil into a mounted collection bin. The soil is analyzed in real-time with a hyperspectral imager for nematode distributions. The soil is repeatedly shaken and analyzed until an appropriate number and distribution of images has been collected for analysis by the machine learning system that predicts soil characteristics. In certain other embodiments the hyperspectral imaging system may take images of the soil or groundcover directly. In still other embodiments, groundcover may be removed from the soil surface and the soil subsequently imaged.

If needed, the system will sample additional locations. For example, the system may start at the edge of a site of suspected contamination and then work towards an area thought to be the source of contamination. Or, samples may be taken randomly or at the dictates of terrain and subsequently correlated to positions within the site.

In certain embodiments, the hyperspectral imager may be part of a hand-held or pack-mounted apparatus. For example, the hyperspectral imaging system may be integrated with a communications device such as a smartphone, tablet, or laptop. The integration may take the form of an electric or otherwise operable connection between an imaging unit and the communications device (e.g., cables, Wi-Fi, Bluetooth, cellular connection, etc.).

In certain embodiments the hyperspectral imager may include one or more additional light sources. The light source may emit one or more spectra of electromagnetic radiation (e.g., a mercury vapor lamp, a calibrated lamp, etc.) or be a relatively monochromatic source (e.g., a laser or LED).

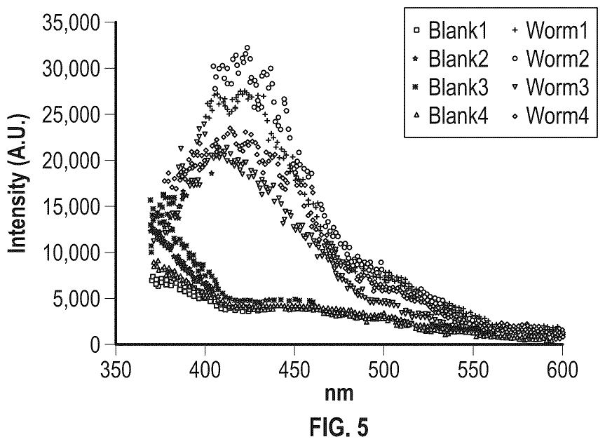

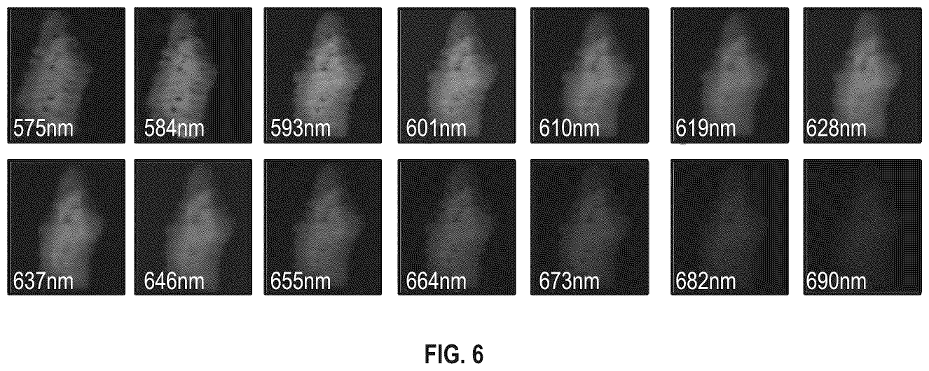

In certain embodiments the light source may emit a wavelength of light capable of triggering autofluorescence in one or more nematode species. By way of non-limiting example, FIG. 5, presents the autofluorescence in 13-day old nematodes measured using a fluorescence spectrophotometer (ex: 340 nm/em: 360 -600 nm). By way of further non-limiting example, FIG. 6 presents a fluorescence scan of the nematode Ascaris lumbricoides at 561 nm excitation.

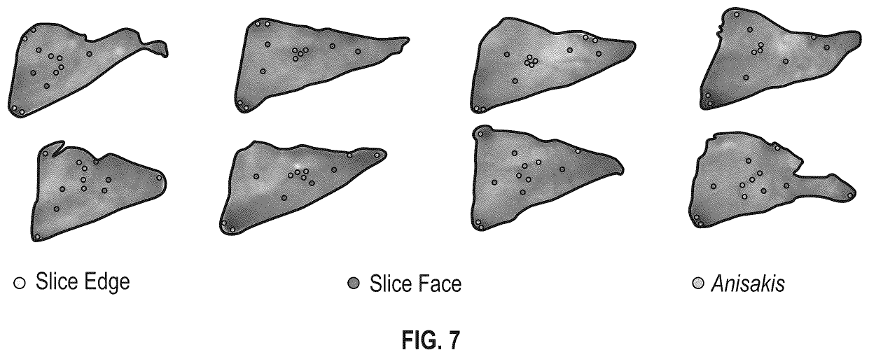

Several methods are commonly used to visualize nematodes. Embodiments of the disclosure focus on visible (Vis) and near infrared (NIR) imaging, alone or in combination with the autofluorescence of the nematode itself. FIG. 7 provides an example of detection of the nematode Anisakis using measurements ranging from 350-1000 nm. Without subscribing or being bound to any theory of operation, it is believed that measurement at wavelengths above 500 nm mitigates soil background interference. Thus, autofluorescence data may be used as part of a training set for a machine learning algorithm employed in embodiments of the disclosure.

In embodiments, images obtained from the hyperspectral imaging system are then passed to the machine learning algorithms to quantify nematode presence in soil samples. As above mentioned, nematodes have shown unique observable responses to organophosphate exposure, including body bend, thrashing, and reduction in locomotion. Envisioned embodiments will track nematode movement to ascertain locomotion changes in response to organophosphate exposure.

Additionally, embodiments of the disclosure are applicable to many different types of bio-indicators and contaminants. Additional envisioned embodiments expand detection to include additional bio-indicators, such as amoebas and bacteria. Including additional bio-indicators would increase the accuracy and sensitivity of the machine learning models by correlating the unique combination of multiple types of microbial life in the sample to a type or level of contaminant. Additional envisioned embodiments further expand the machine learning models to detect more than organophosphates, such as heavy metals in soil which also have morphologically distinct characteristics.

In embodiments building of a training set targeted towards one or more contaminants starts with a clean soil sample split into two samples. One sample is treated with the contaminant (or combinations of contaminants) such as organophosphates. Hyperspectral imaging is then used to detect nematodes and possibly other microbial life (e.g., amoeba, fungi, algae yeasts, bacteria, etc.). The images and corresponding label (treated or untreated) are then inputs used as part of a training set to train a convolutional neural network (CNN) model to identify indicators of organophosphate or selected contaminant levels.

To build a generalized CNN for certain embodiments a test set is built by collecting images as above described. After images are collected, they are post-processed and labeled. In embodiments, edge detection and other techniques, such as the Canny Edge Detector may be used in embodiments of training set data to assist in labeling the images and finding the edges of the nematodes. The CNN will then be trained with the labeled data to create a network model to be run on images automatically and benchmarked against a test set. To get the most accurate predictions, the training set starts with clean images and progressively trains on images with more background noise.

Thus, an alternative embodiment of creating a training data set for use with embodiments of the disclosure may include the following steps. At least one or more nematodes (either a single species or multiple different species) may be exposed to a substance at a predetermined concentration level. The one or more nematodes may then be imaged with the hyperspectral imager and/or additional other imaging systems (e.g., visual cameras, infrared cameras, etc.) to generate a raw image. The raw image may then be processed (e.g., exposure adjusted, resolution adjusted, framing/positioning adjusted, etc.) into a standardized form. The imaging steps may be repeated for various concentrations and substances and/or with various species to build a collection of one or more images. The one or more images may then be assembled (e.g., through labeling, metadata tagging, file system nomenclature setting, etc.) into a training data set. If necessary, generated images may be digitized and/or combined from multiple imaging sources to a single image representative of a set of conditions. The trainding data set may be loaded into a memory storage device (e.g., RAM, ROM, or other physical memory). The memory device may then be interfaced or otherwise operably connected to a device configured to read an interpret the training data set (e.g., a processor, controller, application specific integrated circuit, field programmable gate array, a graphics processing unit, etc.). The training data may then be presented to an input layer of a convoluted neural network or other artificial intelligence structure. One or more parameters of the convoluted neural network or other artificial intelligence structure may then be modified in response to the training data set.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of” shall be closed or semi-closed transitional phrases, respectively.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all of the subject matter of the above description shown in the accompanying drawings shall be interpreted merely as examples illustrating the inventive concept herein and shall not be construed as limiting the disclosure.

Finally, the written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for determining soil contaminant levels, the system comprising:
at least one processor; and,
at least one memory device that stores an application that adapts the at least one processor to:
acquire one or more hyperspectral images from a soil sample provided to a hyperspectral imaging system;
isolate one or more images of nematodes, present in the soil sample;
pass the images of the one or more isolated nematodes into an input layer of a machine learning model trained from a set of known nematode parameters responsive to at least one soil contaminant;
identify and measure bioindicator parameters of the one or more isolated nematodes;
determine a concentration of the at least one soil contaminant; and,
transmit an indicator corresponding to the concentration of the at least one soil contaminant.

2. The system of claim 1 wherein the soil contaminant is at least one of: an organophosphate, an herbicide, a pesticide, a fungicide, and a heavy metal.

3. The system of claim 1 wherein the hyperspectral imaging system includes at least one lighting source configured to induce autofluorescence in nematodes.

4. The system of claim 1 wherein the hyperspectral image is generated in the visible and near-infrared wavelengths.

5. The system of claim 1 wherein the hyperspectral image is generated using wavelengths above 500 nm.

6. The system of claim 1 wherein the nematode parameters include at least one of: presence or absence of hook shaped morphology; nematode count; nematode fluorescence strength and/or color; and nematode motility.

7. The system of claim 1 wherein the machine learning model is a convoluted neural network trained on a training set of nematode parameters.

8. The system of claim 1 wherein the soil sample is imaged in situ.

9. The system of claim 1 further configured to measure at least one or more additional bioindicators.

10. The system of claim 9 wherein the at least one or more additional bioindicators are selected from the group of: amoebas, fungi, algae, yeasts, and bacteria.

11. A method of detecting soil contamination levels comprising:
a) providing a soil sample to a hyperspectral imaging system;
b) generating at least one hyperspectral image with the hyperspectral imaging system;
c) isolating one or more nematode images in the soil sample;
d) passing the images of the one or more isolated nematodes into an input layer of a machine learning model trained from a set of known nematode parameters responsive to a soil contaminant;
e) identifying and measuring the nematode parameters;
f) determining a concentration of the at least one soil contaminant; and,
g) transmitting an indicator corresponding to the concentration of the soil contaminant.

12. The method of claim 11 wherein the soil sample is provided to the hyperspectral imaging system via a sample scoop deployed from an autonomous decontamination system.

13. The method of claim 11 wherein the machine learning model is a convolutional neural network.

14. A non-transitory computer readable medium comprising instructions that adapt at least one processor to:
acquire one or more hyperspectral images from a soil sample provided to a hyperspectral imaging system;
isolate one or more images of nematodes present in the soil sample;
pass the one or more images of isolated nematodes into an input layer of a machine learning model trained from a set of known nematode parameters responsive to a soil contaminant;
identify and measure nematode parameters in the images of the isolated nematodes;
determine a concentration of the at least one soil contaminant; and,
transmit an indicator corresponding to the concentration of the soil contaminant.

15. The non-transitory computer readable medium of claim 14 wherein the machine learning model is an artificial neural network.

16. A method of creating a training data set, comprising;
exposing at least one nematode to a substance at a predetermined concentration level;
imaging the nematode after one or more time intervals generating a raw image;
optionally, processing the raw image;
assembling the one or more images into a training data set.

17. The method of claim 16 further comprising, loading the training data set into a memory storage device.

18. The method of claim 17 further comprising, operably connecting the memory storage device to a device configured to read and interpret the training data set.

19. The method of claim 18 further comprising, presenting the training data set to the input layer of a convoluted neural network.

20. The method of claim 19 further comprising, modifying one or more parameters of the convoluted neural network in response to the training data set.
